(19) United States
(12) Patent Application Publication
(10) Pub. No.: US 2024/0269433 A1
(43) Pub. Date: Aug. 15, 2024

(54) CATHETER INSERTION DEVICE AND METHOD OF INSERTING A CATHETER

(71) Applicant: Becton, Dickinson and Company, Franklin Lakes, NJ (US)

(72) Inventors: Alexander John Segit, Eliot, ME (US); Russell Shepler Cole, River Vale, NJ (US)

(21) Appl. No.: 18/107,866

(22) Filed: Feb. 9, 2023

(51) Int. Cl. A61M 25/01; A61M 25/06

(52) U.S. Cl. CPC A61M 25/0113; A61M 25/0612

(57) ABSTRACT

A catheter insertion device includes a housing, a button including a body and at least one arm extending from the body into the housing to movably couple the button to the housing, the button is movable between a raised position and a depressed position, and a needle hub coupled to the button to move with the button between the raised position and the depressed position. In the raised position, the body of the button is a first distance from the needle hub that reduces visibility of the needle hub through the body of the button. In the depressed position, the body of the button is a second distance from the needle hub less than a first distance that allows visibility of the needle hub through the body of the button.

### TECHNICAL FIELD

The present invention relates generally to medical infusion systems, such as an insulin infusion device or insertion device, where simple, low-profile and low-part count manual insertion device is provided with a dual retraction spring configuration for automatic introducer needle retraction. The dual retraction spring configuration is implemented using multiple barrel-shaped guides and bosses in the insertion device housing which allows for much smaller retraction springs to be used than in a single-barrel configuration.

### BACKGROUND

Diabetes is a group of diseases characterized by high levels of blood glucose resulting from the inability of diabetic patients to maintain proper levels of insulin production when required. Persons with diabetes will require some form of daily insulin therapy to maintain control of their glucose levels. Diabetes can be dangerous to the affected patient if it is not treated, and it can lead to serious health complications and premature death. However, such complications can be minimized by utilizing one or more treatment options to help control the diabetes and reduce the risk of complications.

The treatment options for diabetic patients include specialized diets, oral medications and/or insulin therapy. The main goal of diabetes treatment is to control the diabetic patient's blood glucose or sugar level. However, maintaining proper diabetes management may be complicated because it has to be balanced with the activities of the diabetic patient.

For the treatment of type 1 diabetes, there are two principal methods of daily insulin therapy. In the first method, diabetic patients use syringes or insulin pens to self-inject insulin when needed. This method requires a needle stick for each injection, and the diabetic patient may require three to four injections daily. The syringes and insulin pens that are used to inject insulin are relatively simple to use and cost effective.

Another effective method for insulin therapy and managing diabetes is infusion therapy or infusion pump therapy in which an insulin pump is used. The insulin pump can provide continuous infusion of insulin to a diabetic patient at varying rates to more closely match the functions and behavior of a properly operating pancreas of a non-diabetic person that produces the required insulin, and the insulin pump can help the diabetic patient maintain his/her blood glucose level within target ranges based on the diabetic patient's individual needs.

Infusion pump therapy requires an infusion cannula, typically in the form of an infusion needle or a flexible catheter, that pierces the diabetic patient's skin and through which, infusion of insulin takes place. Infusion pump therapy offers the advantages of continuous infusion of insulin, precision dosing, and programmable delivery schedules.

In infusion therapy, insulin doses are typically administered at a basal rate and in a bolus dose. When insulin is administered at a basal rate, insulin is delivered continuously over 24 hours to maintain the diabetic patient's blood glucose levels in a consistent range between meals and rest, typically at nighttime. Insulin pumps may also be capable of programming the basal rate of insulin to vary according to the different times of the day and night. In contrast, a bolus dose is typically administered when a diabetic patient consumes a meal, and generally provides a single additional insulin injection to balance the consumed carbohydrates. Insulin pumps may be configured to enable the diabetic patient to program the volume of the bolus dose in accordance with the size or type of the meal that is consumed by the diabetic patient. In addition, insulin pumps may also be configured to enable the diabetic patient to infuse a correctional or supplemental bolus dose of insulin to compensate for a low blood glucose level at the time when the diabetic patient is calculating the bolus dose for a particular meal that is to be consumed.

Insulin pumps advantageously deliver insulin over time rather than in single injections, typically resulting in less variation within the blood glucose range that is recommended. In addition, insulin pumps may reduce the number of needle sticks which the diabetic patient must endure, and improve diabetes management to enhance the diabetic patient's quality of life.

Typically, regardless of whether a diabetic patient uses multiple direct injections (MDIs) or a pump, the diabetic patient takes fasting blood glucose medication (FBGM) upon awakening from sleep, and also tests for glucose in the blood during or after each meal to determine whether a correction dose is required. In addition, the diabetic patient may test for glucose in the blood prior to sleeping to determine whether a correction dose is required, for instance, after eating a snack before sleeping.

To facilitate infusion therapy, there are generally two types of insulin pumps, namely, conventional pumps and patch pumps. Conventional pumps require the use of a disposable component, typically referred to as an infusion set, tubing set or pump set, which conveys the insulin from a reservoir within the pump into the skin of the user. The infusion set consists of a pump connector, a length of tubing, and a hub or base from which a cannula, in the form of a hollow metal infusion needle or flexible plastic catheter extends. The base typically has an adhesive that retains the base on the skin surface during use. The cannula can be inserted onto the skin manually or with the aid of a manual or automatic insertion device. The insertion device may be a separate unit required by the user.

Another type of insulin pump is a patch pump. Unlike a conventional infusion pump and infusion set combination, a patch pump is an integrated device that combines most or all of the fluidic components, including the fluid reservoir, pumping mechanism and mechanism for automatically inserting the cannula, in a single housing which is adhesively attached to an infusion site on the patient's skin, and does not require the use of a separate infusion or tubing set. A patch pump containing insulin adheres to the skin and delivers the insulin over a period of time via an integrated subcutaneous cannula. Some patch pumps may wirelessly communicate with a separate controller device (as in one device sold by Insulet Corporation under the brand name OmniPod®), while others are completely self-contained. Such devices are replaced on a frequent basis, such as every three days, when the insulin reservoir is exhausted or complications may otherwise occur, such as restriction in the cannula or the infusion site.

As patch pumps are designed to be a self-contained unit that is worn by the diabetic patient, it is preferable to be as small as possible so that it does not interfere with the activities of the user. Thus, to minimize discomfort to the user, it would be preferable to minimize the overall thickness of the patch pump. However, to minimize the thickness of the patch pump, its constituent parts should be reduced as much as possible. One such part is the insertion mechanism for automatically inserting the cannula into the user's skin.

To minimize the height of the insertion mechanism, some conventional insertion mechanisms are configured to insert the cannula at an acute angle from the surface of the skin, e.g. 30-45 degrees. However, it may be preferable to insert the cannula perpendicular or close to the perpendicular from the surface of the skin, since this would require the minimum length of cannula insertion. In other words, with the minimum length of cannula being inserted into the user's skin, the user can experience greater comfort and fewer complications, such as premature kinking of the cannula. But one problem with insertion of the cannula is determining whether the cannula is fully inserted into the surface of the skin.

Accordingly, a need exists for an improved insertion mechanism for use in a limited space environment, such as in the patch pump, that can cost-effectively insert a cannula vertically or close to perpendicularly into a user's skin, while minimizing or reducing its height and indicating that the cannula is fully inserted into the surface of the skin.

### SUMMARY

In a first aspect, a catheter insertion device includes a housing, a button including a body and at least one arm extending from the body into the housing to movably couple the button to the housing, the button is movable between a raised position and a depressed position, and a needle hub coupled to the button to move with the button between the raised position and the depressed position. In the raised position, the body of the button is a first distance from the needle hub that reduces visibility of the needle hub through the body of the button. In the depressed position, the body of the button is a second distance from the needle hub less than a first distance that allows visibility of the needle hub through the body of the button.

In a second aspect, a catheter insertion device includes a needle hub, and a button movable relative to the needle hub and including a body defining a cavity shaped to be complementary to the shape of the needle hub, and the body has an opacity that restricts visibility of the needle hub through the body when the needle hub is spaced apart from the cavity, and permits visibility of the needle hub through the body when the needle hub is at least partially positioned in the cavity.

In a third aspect, a catheter insertion device includes a button including a body having an first portion and a second portion, the first portion has an opacity that is less than an opacity of the second portion, and a needle hub movable relative to the button, the needle hub includes an indicator that is visible through the second portion and not visible through the first portion when the needle hub is a first distance from the body of the button, and the indicator is visible through both the second portion and the first portion when the needle hub is a second distance from the body of the button, the second distance being less than the first distance.

In a fourth aspect, a catheter insertion device includes: a housing; a button including a body and at least one arm extending from the body into the housing to movably couple the button to the housing, the button is movable between a raised position and a depressed position; and a needle hub movable relative to the button, the needle hub includes an indicator that is visible through the body of the button in the depressed position.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 is an isometric view of an insertion device a pre-activation state in accordance with an embodiment of the present invention;

FIG. 2 is an isometric view of the insertion device of FIG. 1 in a post-activation state in accordance with an embodiment of the present invention;

FIG. 3 is an exploded view of the insertion device of FIG. 1 in accordance with an embodiment of the present invention;

FIG. 4 is an exploded view of a button subassembly 30 of the insertion device of FIG. 1, including a catheter/septum subassembly 10 and an introducer needle subassembly 20, in accordance with an embodiment of the present invention;

FIG. 5 is a view of the completed button subassembly 30 of FIG. 4 in accordance with an embodiment of the present invention;

FIG. 6 is an exploded view of the insertion device and the button subassembly 30 of FIG. 5, with springs within a housing of the insertion device and illustrating the use of temporary protective tubing on the catheter in accordance with an embodiment of the present invention;

FIG. 7A is a cross-sectional view of the insertion device of FIG. 1 in a pre-activation state in accordance with an embodiment of the present invention;

FIG. 7B is another cross-sectional view of the insertion device of FIG. 1 in a pre-activation state in accordance with an embodiment of the present invention;

FIG. 8 is a transparent view of the insertion device of FIG. 1 in the pre-activation state illustrating a position of a radial operation pin within a helical pathway in accordance with an embodiment of the present invention;

FIG. 9 is a transparent view of the insertion device of FIG. 1 in an intermediate activation state illustrating a position of the radial operation pin in accordance with an embodiment of the present invention;

FIG. 10 is a cross-sectional view of the insertion device of FIG. 1 in a post-activation state in accordance with an embodiment of the present invention;

FIG. 11 is a transparent view of the insertion device of FIG. 1 at the post-activation state illustrating a position of the radial operation pin at full retraction in accordance with an embodiment of the present invention;

FIG. 12 is a perspective view of a patch pump incorporating a low-profile cannula insertion device, illustrated without a cover for clarity;

FIG. 13 is an exploded view of the various components of the patch pump of FIG. 12, illustrated with a cover;

FIG. 14 is a perspective view of an alternative design for a patch pump having a flexible reservoir, illustrated without a cover; and

FIG. 15 is a patch-pump fluidic architecture and metering sub-system diagram of the patch pump of FIG. 14.

### DETAILED DESCRIPTION

The exemplary embodiments of the present invention described below provide novel means of providing one or more infusion device elements that are configured to insert a catheter into skin of a user and indicate when the catheter is fully inserted into the skin, but embodiments are not limited thereto. The insertion device is configured to perform a manual insertion of the catheter which allows the insertion device to be smaller, simpler and cheaper than automatic or spring-assisted insertion devices.

Exemplary embodiments of the present invention described below utilize a manual insertion device and include a dual retraction spring configuration for automatic introducer needle retraction that also allows for a very small device size. The dual retraction spring configuration is implemented using a plurality of cylindrical or barrel-shaped guides. In an exemplary embodiment, one barrel guides a button and catheter, and adjacent barrels house retraction springs, one on each side of the button and catheter. Having the springs in separate barrels allows for much smaller springs than a single-barrel configuration in which the spring is coaxial with the catheter. A single coaxial spring creates access to the button assembly since spring design limitations require the spring to extend nearly from the bottom of the housing to the top. Access is required for features like the locking arm and, if the features are implemented inside the spring, the entire mechanism must grow to accommodate them, increasing the mechanism foot print.

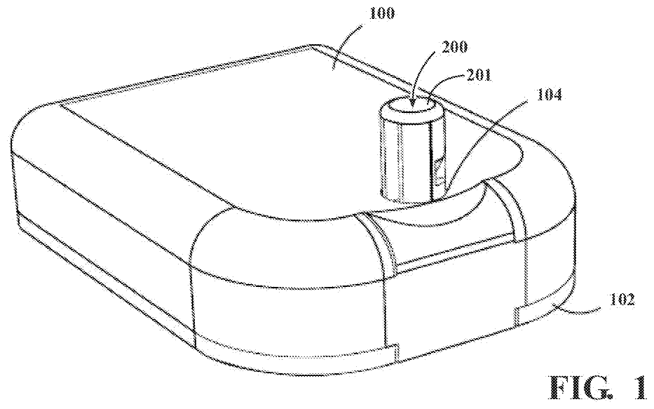

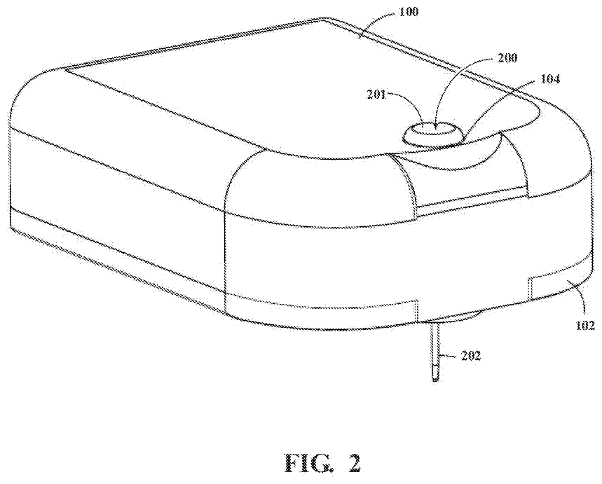

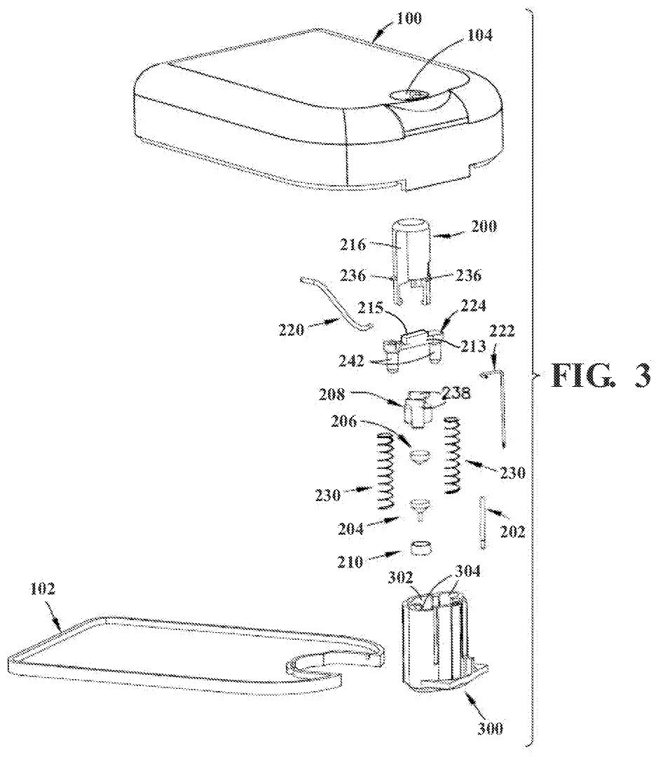

FIG. 1 shows the insertion device before use and FIG. 2 shows the device after deployment of the cannula. The insertion device includes a top housing 100 and a base 102. The top housing 100 is shown having an opening 104 through a top surface from which a user-accessible, and user-acutatable button 200 slidably extends. The content of the insertion device, including the mechanism housing 300, is shown in greater detail in FIG. 3. The top housing 100, button 200, and mechanism housing 300 can be manufactured from ABS, and the base 102 can be manufactured from PETG, but embodiments are not limited thereto.

As shown in FIG. 3, the exemplary insertion device is assembled by stacking together a number of subassemblies which are trapped between the top housing 100 and the mechanism housing 300. The subassemblies shown in FIG. 3 and discussed in greater detail below include a catheter/septum subassembly 10, an introducer needle subassembly 20, and a button subassembly 30. Other features and functions of the insertion device that are well-known to those skilled in the art are omitted from the figures and discussion for clarity.

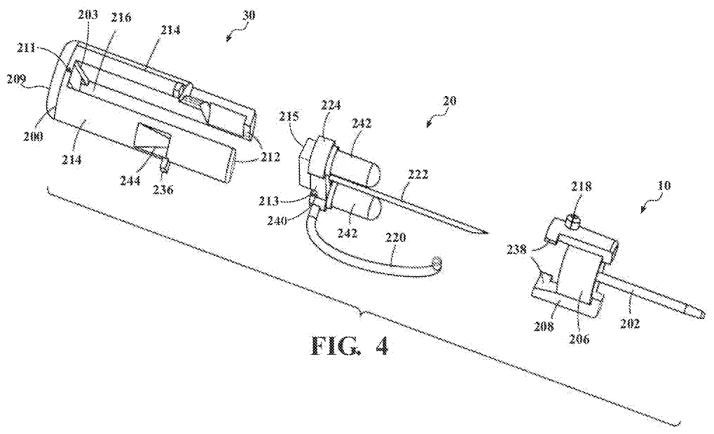

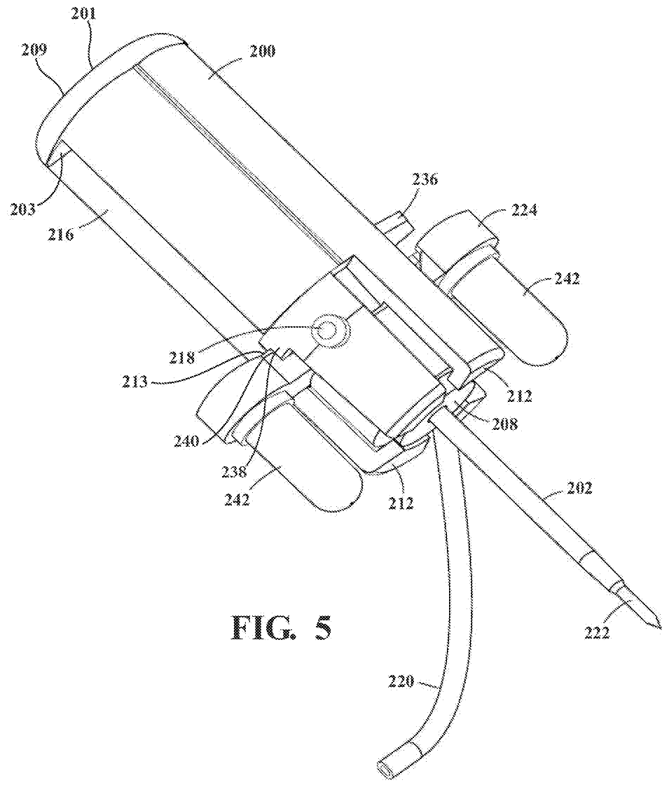

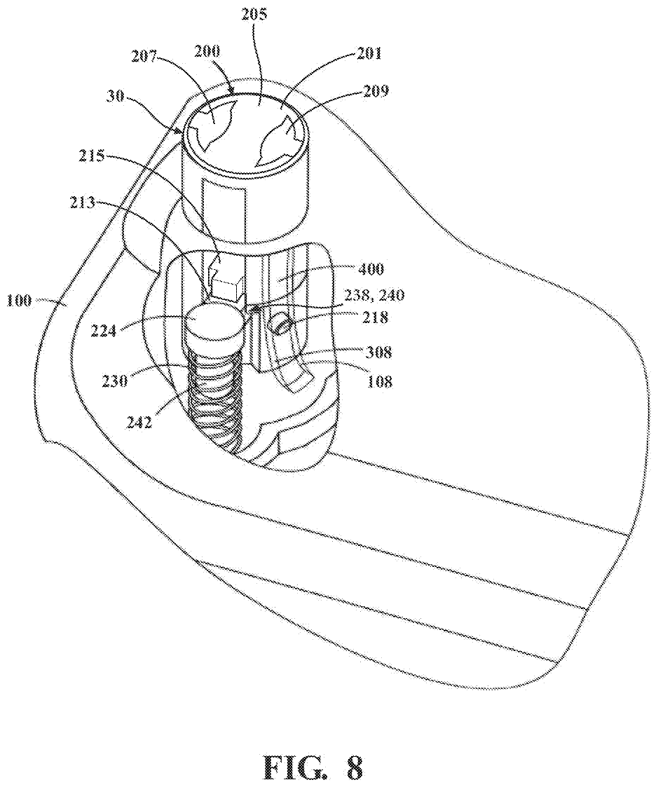

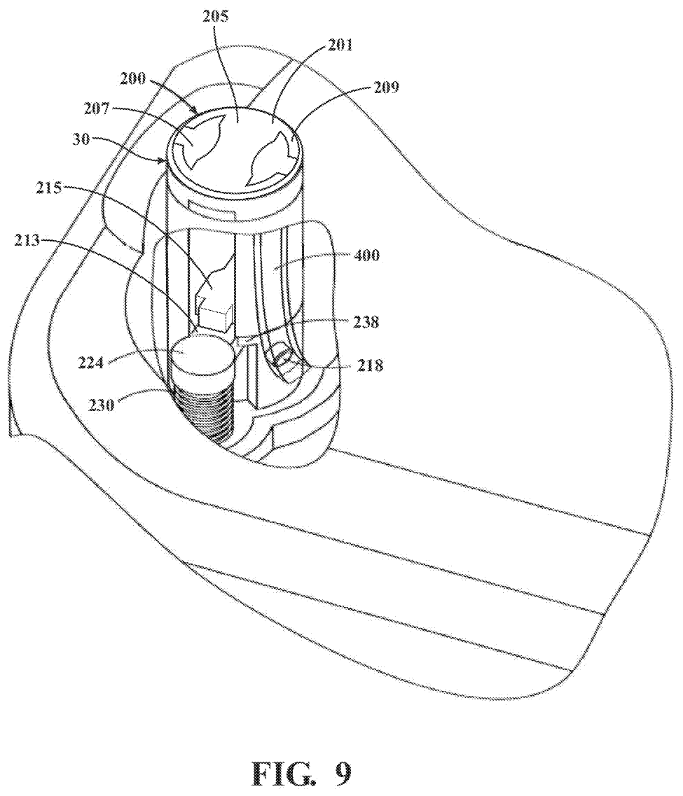

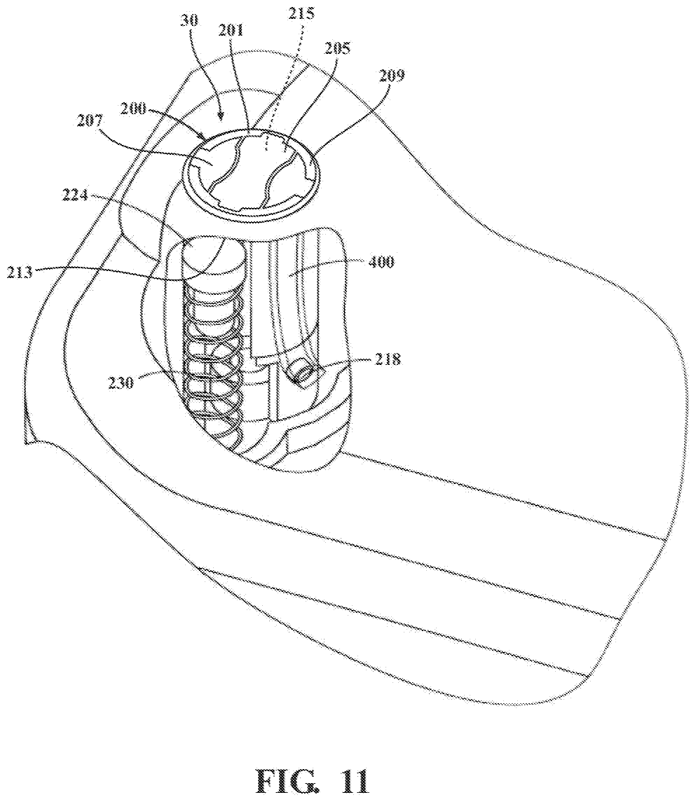

An exemplary button subassembly 30 is shown in FIG. 4, including the catheter/septum subassembly 10 and introducer needle subassembly 20, and FIG. 5 depicts the completed button subassembly 30 of the insertion device of FIG. 1 in accordance with an embodiment of the present invention. The button subassembly 30 is built by combining the catheter/septum subassembly 10 and introducer needle subassembly 20 with the button 200. As described in greater detail below, once assembled, the introducer needle subassembly 20 cannot be rotated in the button 200. The catheter/septum subassembly 10 can be rotated in the button 200 and in doing so, can be rotated from a position secured with the introducer needle subassembly 20, to a position freed from the introducer needle subassembly 20. For example, the button subassembly 30 may be movable between a pre-activation state (FIG. 8), an intermediate state (FIG. 9), and a post-activation state (FIG. 11), where in the pre-activation state, the button 200 of the button subassembly 30 is in a raised position, in the intermediate state, the button 200 is in an intermediate position, and in the post-activation state, the button 200 is in a depressed position. Each of the states and positions will be described in greater detail below.

Specifically, according to one embodiment, the button subassembly 30 is built by inserting the introducer needle 222 of the introducer needle subassembly 20 through the septum 206 and catheter 202 of the catheter/septum subassembly 10, or catheter hub 10. The catheter/septum subassembly 10 is then secured to the introducer needle subassembly 20 by rotating the catheter/septum subassembly 10 up to about 20 degrees or more to lock the detents or teeth 238 on the release collar 208 into grooves, slots, or detents 240 on the top surface of the introducer needle hub 224, which couples the introducer needle hub 224 and catheter/septum subassembly 10. In this position the teeth 238 are locked over the top the introducer needle hub 224 so as the button 200 is pressed down, the introducer needle hub 224 also moves down. This results in the introducer needle 222 and catheter 202 being moved simultaneously for insertion into a user's skin (not shown). The selective coupling of the needle hub 224 to the collar 208 through engagement with the teeth 238 permits the movement of the catheter/septum subassembly 10 with the needle hub 224 and the button 200.

The button subassembly 30 is then completed by snapping the release collar 208 into the button 200 to secure the introducer needle subassembly 20 and the catheter/septum subassembly 10 in place. To do so, the button 200 can include detents 212 on deflectable arms 214 to deflect and then capture therebetween the lower edge of the release collar 208 as shown in FIG. 5. Between the deflectable arms 214, slots 216 are provided in the button 200 to allow linear travel of the introducer needle hub 224 relative to the button 200, but prohibit rotational movement of the introducer needle hub relative to the button 200. The slots 216 in the button 200 also allow rotational movement of the radial operation pin 218 of the release collar 208 relative to the button 200 as described in greater detail below. In the exemplary embodiment, a substantially cylindrical-shaped pin 218 is shown on an outer circumference of the release collar 208. However, in this or other embodiments of the present invention, any detent or projection of the release collar which can operate with the helical pathway can be provided as the radial operation pin.

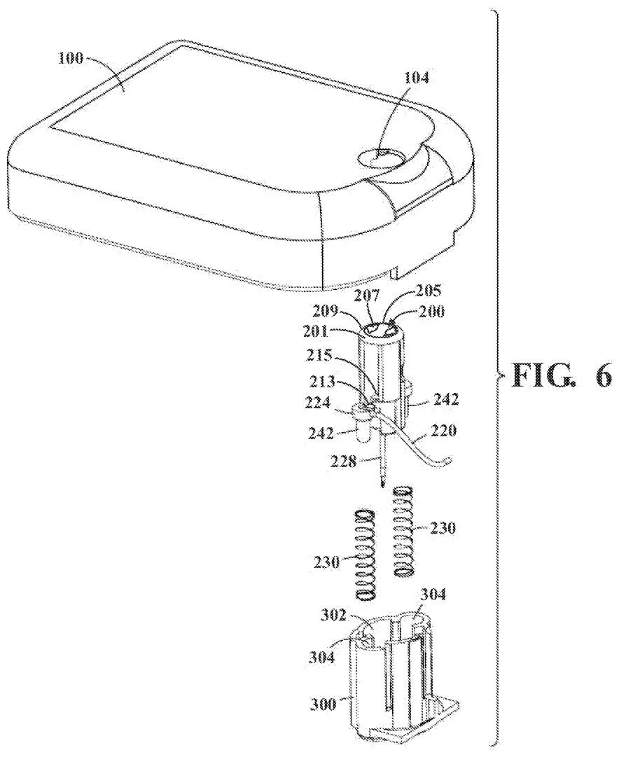

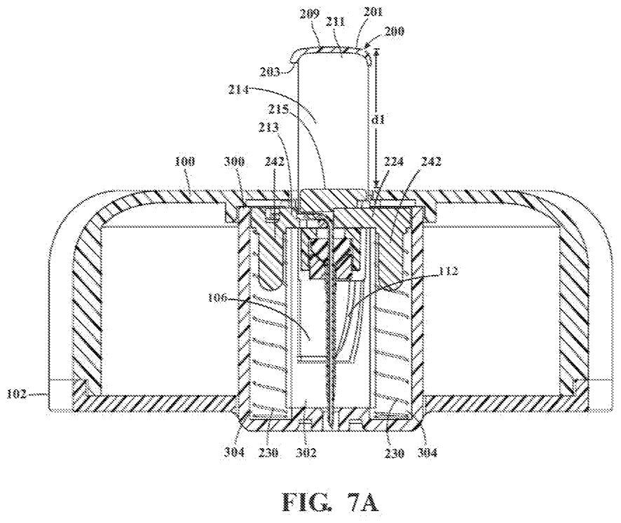

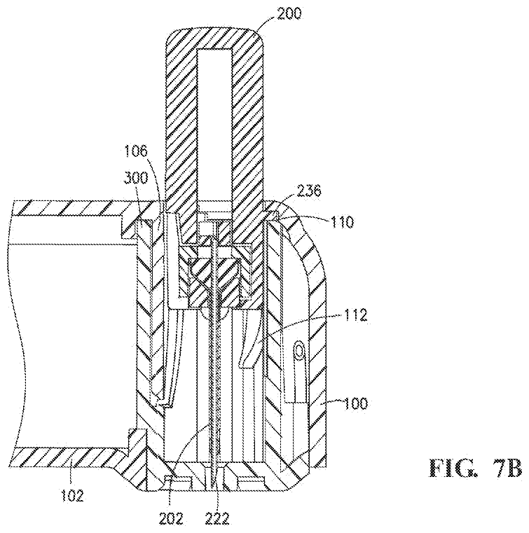

The button subassembly 30 can then be assembled with the housing top 100 and mechanism housing 300. FIG. 6 is a view of the assembly of the button subassembly 30 and springs into the housing of the insertion device of FIG. 1 and illustrating the use of temporary protective tubing on the catheter. To complete assembly, the button 200 and assembly thereof is slidably assembled with a projection 106 extending from an inner surface of the top housing 100 as shown in greater detail in FIG. 7A. FIG. 7A is a sectional view of the fully assembled insertion device of FIG. 1 in a pre-activation state in accordance with an embodiment of the present invention. A button lock arm 112 of the top housing 100 retains the button subassembly 30 in place during the next assembly step which is placing the mechanism housing 300 into the top housing 100 thereby trapping the other subassemblies therein.

The mechanism housing 300 is preferably comprised of three cylinders, guides or barrels, including a center barrel 302 that slidably receives and guides the button subassembly 30, and two barrels 304, one on each side of the center barrel 302 that constrain the springs 230. During assembly, the springs 230 are captured between bosses 242 of the introducer needle hub 224 and a bottom of the barrels 304 of the mechanism housing 300. In doing so, the springs 230 exert an expansion force between the introducer needle hub 224 and a bottom of the barrels 304 of the mechanism housing 300. In the exemplary embodiment, a plurality of springs 230 and adjacent barrels 304 are shown. However, in this or other embodiments of the present invention, a single spring and adjacent barrel can be provided in substantially the same manner, wherein the unused adjacent barrel can be left empty or can be omitted entirely. Still further, a single spring can be provided in the button top and extended during insertion that, upon completion, retracts to its natural state thereby retracting the introducer needle from the catheter.

The rounded bosses 242 are provided with a diameter and length to center and align the springs 230 during operation. The springs 230 can be partially preloaded during assembly of the insertion device, and the mechanism housing 300 can be laser welded or glued to the top housing 100. The bottom or base 102 can then be added. In doing so, the full and complete insertion mechanism subassembly can be placed onto the base 102 with all of the other components, as the last assembly step. Having the completed insertion mechanism subassembly allows for easy handling in production, as opposed to trapping all of the parts between the top and bottom housings. In an exemplary production, the mechanism housing 300 would be attached to the top housing 100 using snaps or adhesive (not shown) which holds together the mechanism.

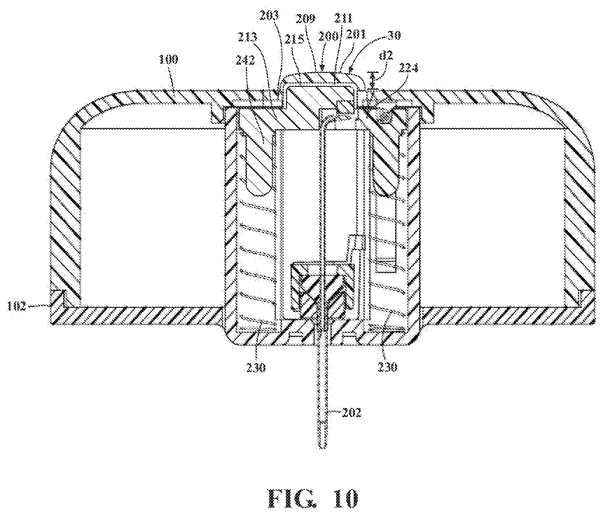

Referring still to FIG. 7A, the fully assembled insertion device is depicted in the pre-activation state in accordance with an embodiment of the present invention. As shown in FIG. 7A, the arms 214 of the button 200 extend from an underside 203 of a body 201 of the button 200 into the housing 100 to movably couple the button 200 to the housing 100, permitting the button 200 to be movable between the raised position and the depressed position (FIG. 10). The body 201 may include a first portion 205 and a second portion 207 at an upper side 209 of the body 201 opposite the underside 203, where the first portion 205 has an opacity that is less than an opacity of the second portion 207. However, it is contemplated and possible that the first portion 205 and the second portion 207 each have opacities that are the same. The first portion 205 may be formed by any traditional method or material for increasing an opacity of the body 201, such as frosted plastic or glass, bruting, or the like. As used herein, bruting is a process of increasing a roughness of a surface for increasing opacity by, for example, sanding. The body 201 may define a cavity 211 formed in the underside 203 of the button 200 and shaped to be complementary to the shape of the needle hub 224 to be configured to at least partially enclose the needle hub 224 in the depressed position.

The first portion 205 may have an opacity that restricts visibility of the needle hub 224 through the body 201 when the needle hub 224 is spaced apart from the cavity 211, and permits visibility of the needle hub 224 through the body 201 when the needle hub 224 is at least partially positioned in the cavity 211. The first portion 205 is at least partially positioned directly opposite the cavity 211 such that the needle hub 224 is visible through the first portion 205 when at least partially positioned within the cavity 211. As will be described in greater detail below, the needle hub 224 may be positioned within the cavity 211 when the button 200 is in the depressed position (FIG. 10). The first portion 205 may be positioned closer to the cavity 211 than the second portion 207 so that the needle hub 224 may only be visible through the first portion 205, and not the second portion 207, when the needle hub 224 is positioned within the cavity 211. In other words, an orthographic projection of the cavity 211 extending perpendicularly through the underside 203 at least partially intersects the first portion 205.

As discussed above, the needle hub is coupled to the button to move with the button between the raised position and the depressed position. As shown in FIG. 7A, the needle hub includes a base 213 and an indicator 215 that extends from the base 213 toward the underside 203 of the body 201 of the button 200. The indicator 215 may be formed of a material or include a color or marking that is visible through the second portion 207 and not visible through the first portion 205 when the needle hub 224 is a first distance d1 from the body 201 of the button 200, and is visible through both the second portion 207 and the first portion 205 when the needle hub 224 is a second distance d2 from the body 201 of the button 200, where the second distance d2 is less than the first distance d1. For example, the indicator 215 may include a traditional high-visibility color, such as orange, or a fluorescent color. The indicator 215 may only be visible through the body 201 of the button 200 when the button 200 is in the depressed position.

Referring to FIGS. 7A and 7B, according to one embodiment, one or more breakable ribs 236 (FIGS. 4 and 5) on the activation button 200 are captured by step detents 110 in the top housing 100 to hold the button 200 in the pre-activation state. A safety tab (not shown) could also be positioned in the button slot which would prevent accidental activation during shipping and handling of the device once it is removed from the packaging. The safety tab would be removed just prior to insertion.

Referring to FIGS. 7A-8, in the pre-activation state with the button 200 in the raised position, the body 201 of the button 200 is spaced apart the first distance d1 from the needle hub 224. As will be described in greater detail below, when the body 201 of the button 200 is spaced apart the first distance d1 from the needle hub 224, the body 201 restricts visibility of the needle hub 224 through the body 201 so that the needle hub 224 is not clearly visible through the body 201.

To activate the device, the user pushes the button 200 into the top housing 100. Once the ribs 236 break or a deformation force threshold is exceeded, the three ribs 236 yield and the button 200 abruptly moves downward inserting the introducer needle 222 and catheter 202, and loading the retraction springs 230. The springs 230 can be partially preloaded during assembly of the insertion device. The minimum break force of the breakable ribs 236 ensures that the user pushes hard enough to fully insert the catheter. Partial activation would result in the catheter not fully inserting, the introducer needle not retracting and the catheter not locking in the post activation position.

The release of the button 200 from the ribs 236 is configured to occur once a desired amount of activation force has been applied to the button 200. Since the button 200 is releasably held in the raised position by the engagement between the ribs 236 and the step detents 110, the force applied to the button 200 by the user steadily increases for some period of time prior to release. Upon sudden release, the force upon the button 200 has reached a desired value and therefore, the button 200 is accelerated downward due to the sudden freedom to travel and the desired force applied to the button 200 at the time of release and maintained thereafter. Such release ensures that a desired amount of downward force, speed, smoothness and angle has been applied by the user. Such activation substantially eliminates variations in the user force applied, speed, smoothness and angle thereof, and reduces insertion failure and/or discomfort to the user.

Referring to FIGS. 9-11, after the release of the button 200, the button subassembly 30 and components therein begin to travel through the mechanism housing 300. One of the two teeth 238 on the release collar 208 (see, e.g., FIG. 4) couples the introducer needle hub 224 and catheter/septum subassemblies 10. In this position the teeth 238 are locked over the top the introducer needle hub 224 so as the button 200 is pressed down, the introducer needle hub 224 moves down as well. As the button 200 is pressed down, the introducer needle hub 224 moves down as well, which results in the introducer needle 222 and catheter 202 being simultaneously inserted into a user's skin (not shown), and also results in the introducer needle hub 224 compressing the springs 230, or biasing member 230, engaged with the needle hub 224. When compressed, the springs 230 bias the needle hub 224 toward the body 201 of the button 200. To create an insertion device with a small foot print, each of the springs 230 has a small diameter relative to the compression length which, if unsupported, would cause the springs to buckle during compression. The bosses 242 on the introducer needle hub 224 translate through the middle of the springs 230 during compression to prevent the springs 230 from buckling. In the exemplary embodiment, the springs 230 are compressed, and exert an expansion force to retract the introducer needle hub and introducer needle. However, in this or other embodiments of the present invention, one or more extension springs can be used, and exert a retraction force to retract the introducer needle hub and introducer needle.

As noted above, the catheter/septum subassembly 10 is attached to the button 200 and introducer needle hub 224 but is free to rotate up to about 20 degrees around the primary axis. In this case, the primary axis is defined as the axis extending along the geometric center of the insertion needle 222. Slots 216 are provided in the button 200 to allow linear travel of the introducer needle hub 224 relative to the button 200, but prohibit rotational movement of the introducer needle hub relative to the button 200. The slots 216 in the button 200 also allow rotational movement of the radial operation pin 218 of the release collar 208 relative to the button 200. The angle of this rotation is controlled by the radial operation pin 218 extending from the release collar 208. During insertion, that is, downward travel of the button subassembly 30, the radial operation pin 218 travels in a helical pathway 400, or slot 400, defined by the housing 100 and created by the combined features in the top housing 100 and mechanism housing 300. During such travel, the radial operation pin 218 of the release collar 208 rotates, or pivots, the release collar 208 of the catheter hub 10 when the catheter hub 10 moves with the button 200 between the raised position and the intermediate position, eventually releasing the introducer needle subassembly 20 from the catheter/septum subassembly 10 when the button 200 is in the intermediate position. The surfaces 108 in the top housing 100, and 308 in the mechanism housing 300 that create the helical pathway 400 are divided between two parts, so that both parts can be molded without slides. That is, by creating the helical pathway 400 using the coupling of two separately molded parts, a single part having the slide or pathway molded therein is not required, significantly simplifying the manufacture of the insertion device. FIGS. 8, 9, and 11 show the surface 108 in the top housing 100, and 308 in the mechanism housing 300 that create the helical pathway 400 when assembled.

As noted above, the slots 216 provided in the button 200 allow movement of the radial operation pin 218 of the release collar 208. Further, the catheter/septum subassembly 10 is attached to the button 200 and introducer needle hub 224, but is free to rotate up to 20 degrees around the primary axis. Such 20 degrees of rotation permits the travel of the radial operation pin 218 of the release collar 208 in the helical pathway 400. As the button 200 is pressed down, the release collar 208 and radial operation pin 218 of the release collar 208 move down as well through the stationary top housing 100 and mechanism housing 300. The radial operation pin 218 of the release collar 208 therefore, slidably disposed in the helical pathway 400, rotates the release collar when moved down through the stationary top housing 100 and mechanism housing 300 by the button 200.

In the pre-activation state, the radial operation pin 218 angle is constrained to an orientation in which the teeth 238 of the release collar 208 are fully engaged with the introducer needle hub 224. During button 200 movement between the pre-activation state and the post-activation state, the radial operation pin 218 of the release collar 208 rotates the release collar 208 when moved through helical pathway 400 of the stationary top housing 100 and mechanism housing 300.

In the post-activation state, the radial operation pin 218 has been rotated up to about 20 degrees, which decouples the introducer needle hub 224 from the teeth 238 of the release collar 208, freeing the introducer needle hub 224 from the release collar 208, to be retracted by the compressed springs 230. The release collar 208 and other elements of the catheter/septum subassembly 10 are left in the down and inserted position.

FIG. 9 shows the insertion device during insertion of the introducer needle 222 and catheter 202, and in an intermediate state just before the introducer needle hub 224 is released by the radial operation pin 218 of the release collar 208 for retraction. In the intermediate state, the button 200 is in an intermediate position between the raised position (FIGS. 7 and 8) and the depressed position (FIGS. 10 and 11). In the intermediate state with the button 200 in the intermediate position, the introducer needle 222 and the catheter 202 are extended from the housing 100 to be inserted into a user's skin, but prior to retraction of the needle hub 224 relative to the button 200. In this position, the catheter 202 surrounds the needle 222 to be extended with the needle 222 in the intermediate state. When moving from the raised position to the intermediate position, the needle 222 moves with the button 200 to extend out of the housing 100, and when moving from the intermediate position to the depressed position, the needle 222 retracts toward the underside 203 of the body 201 of the button 200.

In the intermediate state, the radial operation pin 218 and the release collar 208 are almost fully rotated by engagement with the helical pathway 400. At the end of rotation by the helical pathway 400, the teeth 238 on the release collar 208 are about to move free of the detents 240 (see, e.g., FIGS. 4 and 5) of the introducer needle hub 224 and release the introducer needle hub 224 so it can be pushed up and retracted by the springs 230. That is, as the radial operation pin 218 and the release collar 208 are rotated by engagement with the helical pathway 400, the teeth 238 on the release collar 208 simultaneous rotate until free of the detents 240 of the introducer needle hub 224.

At this point, the release collar 208 being held down by the button 200 is no longer secured to the introducer needle hub 224, and the springs 230 force the introducer needle hub 224 and introducer needle 222 upward and into the retracted position, leaving the catheter/septum subassembly 10 in the down and inserted position. The button 200 is locked in the down position, thereby holding the catheter/septum subassembly 10 in the down and inserted position. The lock arm 112 that protrudes from the top housing 100 that retains the button subassembly 30 in place during assembly can also be configured to snap into a detent 244 in the button 200 in the post-activation state locking the button subassembly 30 in place keeping the catheter in the skin.

Referring still to FIG. 9, the retraction springs 230 are fully compressed and the radial operation pin 218 and release collar 208 have been rotated to an extent required for decoupling the teeth 238 of the release collar 208 from the introducer needle hub 224 to release the introducer needle hub 224 for retraction.

Referring to FIGS. 10 and 11, the insertion device is depicted in the post-activation state with the button 200 in the depressed position. In the post-activation state, the button 200 is fully depressed in the depressed position with the catheter 202 positioned in a user's skin. The biasing member 230 biases the introducer needle 222 to retract farther into the housing 100 than its pre-activation state to ensure needle stick shielding and to protect the catheter 202 from damage. The tip of the introducer needle 222 remains sealed by the septum 206 in the fluid path to form an uninterrupted fluid path with the catheter 202. In this or other embodiments, the tip or distal portion of the introducer needle 222 remains within the catheter 202. When the introducer needle 222 retracts into the housing 100, the needle hub 224 is retracted toward the body 201 of the button 200 with the indicator 215 at least partially positioned within the cavity 211 of the button 200 to be visible through the body 201. When the button 200 is in the depressed position, the body 201 of the button 200 is a second distance d2 from the needle hub 224 with the needle hub 224 adjacent the body 201, the second distance d2 being less than the first distance d1 that allows the needle hub 224 to be visible through the body 201 of the button 200. As shown in FIG. 11, when the insertion device is in the post-activation state, the indicator 215 is visible through the body 201 of the button 200.

In the exemplary embodiments, manual insertion of the introducer needle and catheter allows the insertion device to be smaller, simpler and cheaper than insertion devices employing spring assisted insertion. Other patch pump plastic catheter insertion mechanisms use insertion springs which are large relative to the retraction spring because the insertion force is large relative to the retraction force. Fully integrated, spring assisted insertion also allows for angled insertion for a low profile device, which increases the stroke and greatly increases the wound and mechanism size. The insertion spring serves no purpose after insertion, but simply takes up room in the device wherein size is one of the most important user requirements for the product.

In the exemplary embodiments, the dual retraction spring configuration also allows for a very small size. One barrel of the insertion device housing guides the button and catheter, and the adjacent barrels house the two retraction springs. Having the springs in separate barrels and directed by bosses on the introducer needle hub allows for much smaller springs than a single barrel configuration in which the spring is coaxial with the catheter. A single coaxial spring creates access to the button assembly since spring design limitations require the spring to extend nearly from the bottom of the housing to the top. Access is required for features like the locking arm and if the features are implemented inside the spring, the entire mechanism must grow to accommodate them increasing the mechanism foot print. Passively locking the catheter down and retracting the introducer needle creates the simplest possible manual insertion user interface for a manual insertion mechanism which is a single button push.

As noted, the retraction springs 230 are minimally loaded before use to ensure that the introducer needle 222 retracts into the device completely. The springs 230 load further during insertion. Providing minimally loaded springs and not fully loaded springs in the insertion device, reduces the risk associated with sterilizing and storing loaded springs and simplifies the design.

To operate the insertion device, the user applies the insertion device to a user's skin using an adhesive upon the base 102 of the device. The user then manually pushes the protruding button 200 until breaking or deforming the ribs 236. The button 200, now suddenly free to travel, is rapidly pushed into the top housing 100 and serves to push and insert the plastic catheter 202 and introducer needle 222 into a user's skin. As the button 200 is being pushed, the release collar 208 is rotated by the radial operation pin 218 of the release collar 208 moving through helical pathway 400. The release collar 208 is rotated to an extent required for decoupling the release collar 208 from the introducer needle hub 224, and the introducer needle hub 224 and introducer needle 222 are then retracted to a retracted position, exceeding that of the original needle position to ensure needle shielding. The plastic catheter 202 now uncoupled from the introducer needle 222 is left in the down and inserted position. The button 200 automatically locks in the down position, flush with the top of the housing, which also locks the catheter at the desired depth, for example, in the subcutaneous layer. A sensor (not shown) can be provided to sense the post-activation state and advise other electronics (not shown) that the catheter has been inserted properly which allows the patient to infuse medicament. A pump or reservoir then infuses medicament through the introducer needle, into the catheter and out into the patient's subcutaneous layer. To best target the desired depth, the base can include skin interface geometry to achieve and maintain a desired insertion depth, avoid skin tenting, and/or tension the skin at the insertion site.

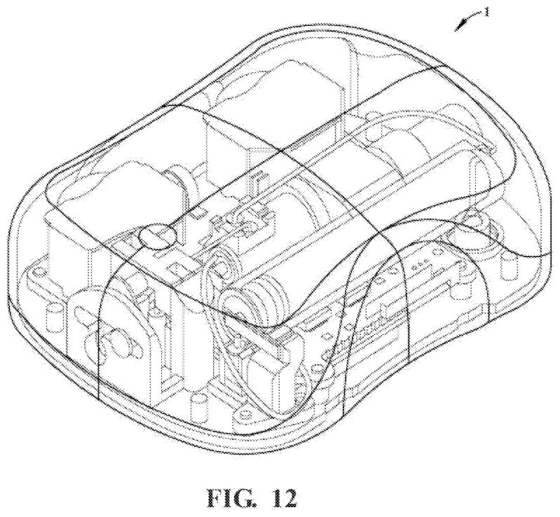

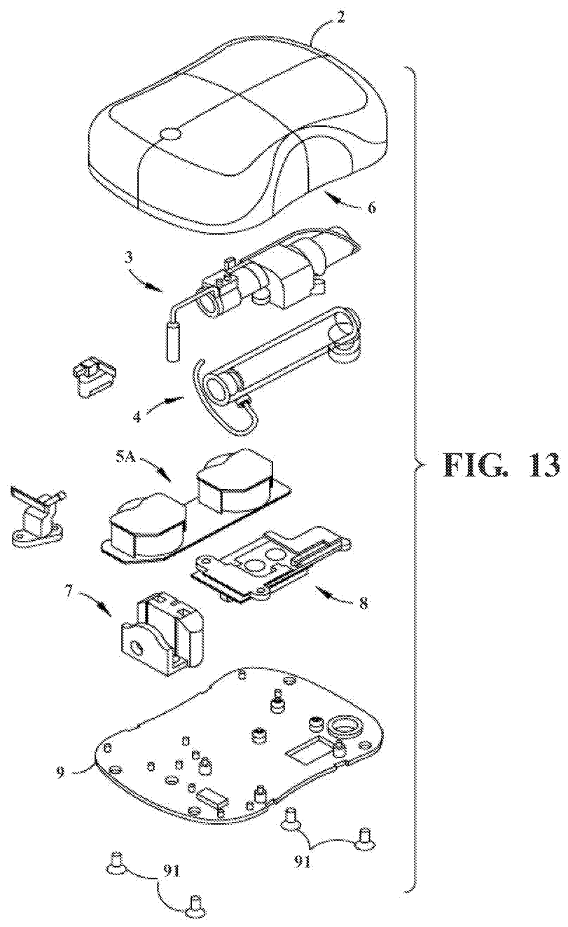

In the above embodiments, a patch pump can be provided with one or more of the described features. FIG. 12 is a perspective view of an exemplary embodiment of a patch pump 1 according to an exemplary embodiment of the invention. The patch pump 1 is illustrated with a see-through cover for clarity and illustrates various components that are assembled to form the patch pump 1. FIG. 13 is a view of the various components of the patch pump of FIG. 12, illustrated with a solid cover 2. The various components of the patch pump 1 may include: a reservoir 4 for storing insulin; a pump 3 for pumping insulin out of the reservoir 4; a power source 5 in the form of one or more batteries; an insertion mechanism 7 for inserting an inserter needle with a catheter into a user's skin; control electronics 8 in the form of a circuit board with optional communications capabilities to outside devices such as a remote controller and computer, including a smart phone; a dose button 6 on the cover 2 for actuating an insulin dose, including a bolus dose; and a base 9 to which various components above may be attached via fasteners 91. The patch pump 1 also includes various fluid connector lines that transfer insulin pumped out of the reservoir 4 to the infusion site.

As noted above, it should be understood that inserter mechanisms come in various configurations. In some embodiments, the inserter mechanism inserts a soft catheter into the skin. In these embodiments, typically the soft catheter is supported on a rigid insertion needle. The insertion needle is inserted into the skin along with the soft catheter, and then retracted from the skin, leaving the soft catheter in the skin. In other embodiments, a soft catheter is not provided, and the insertion needle remains in the skin and forms a portion of the insulin flow path to deliver insulin until the infusion is finished. Insertion needles are typically hollow, and need to be hollow if they form part of the insulin flow path. However, insertion needles that support a soft catheter and then retract may be solid or hollow. If the insertion needle deploys a soft catheter, and retracts but remains part of the insulin flow path, then the insertion needle should be hollow. However, if the insertion needle deploys a soft catheter and then retracts but does not form part of the insulin flow path, then the insertion needle may be solid or hollow. In either case, the insertion needle is preferably rigid enough to reliably penetrate the skin, but otherwise may be made flexible enough to provide comfort to the user.

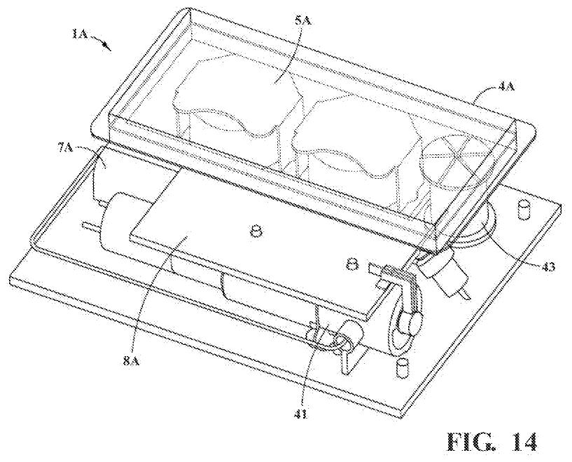

FIG. 14 is a perspective view of an alternative design for a patch pump 1A having a flexible reservoir 4A, and illustrated without a cover. Such arrangement may further reduce the external dimensions of the patch pump 1A, with the flexible reservoir 4A filling voids within the patch pump 1A. The patch pump 1A is illustrated with a conventional cannula insertion device 7A that inserts the cannula, typically at an acute angle, less than 90 degrees, at the surface of a user's skin. The patch pump 1A further comprises: a power source 5A in the form of batteries; a metering sub-system 41 that monitors the volume of insulin and includes a low volume detecting ability; control electronics 8A for controlling the components of the device; and a reservoir fill port 43 for receiving a refill syringe 45 to fill the reservoir 4A.

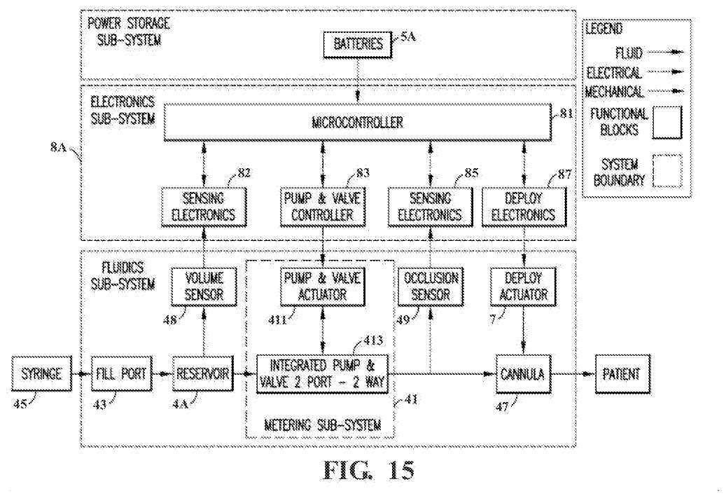

FIG. 15 is a patch-pump fluidic architecture and metering sub-system diagram of the patch pump 1A of FIG. 14. The power storage sub-system for the patch pump 1A includes batteries 5A. The control electronics 8A of the patch pump 1A may include a microcontroller 81, sensing electronics 82, pump and valve controller 83, sensing electronics 85, and deployment electronics 87 that control the actuation of the patch pump 1A. The patch pump 1A includes a fluidics sub-system that may include a reservoir 4A, volume sensor 48 for the reservoir 4A, a reservoir fill port 43 for receiving a refill syringe 45 to refill the reservoir 4A. The fluidics sub-system may include a metering system comprising a pump and valve actuator 411 and an integrated pump and valve mechanism 413. The fluidics sub-system may further include an occlusion sensor 49, a deploy actuator 7, as well as the cannula 47 for insertion into an infusion site on the user's skin. The architecture for the patch pumps of FIGS. 12 and 13 is the same or similar to that which is illustrated in FIG. 15.

Although only a few exemplary embodiments of the present invention have been described in detail above, those skilled in the art will readily appreciate that many modifications are possible in the exemplary embodiments without materially departing from the novel teachings and advantages of this invention. Any of the embodiments and/or elements disclosed herein may be combined with one another to form various additional embodiments not specifically disclosed, as long as they do not contradict each other. It is particularly noted that those skilled in the art can readily combine the various technical aspects of the various elements of the various exemplary embodiments that have been described above in numerous other ways, all of which are considered to be within the scope of the invention, which is defined by the appended claims and their equivalents

What is claimed is:

1. A catheter insertion device comprising:
a housing;
a button comprising a body and at least one arm extending from the body into the housing to movably couple the button to the housing, the button is movable between a raised position and a depressed position;
a needle hub coupled to the button to move with the button between the raised position and the depressed position,
in the raised position, the body of the button is a first distance from the needle hub that reduces visibility of the needle hub through the body of the button, and
in the depressed position, the body of the button is a second distance from the needle hub, the second distance being less than the first distance, and allowing visibility of the needle hub through the body of the button.

2. The catheter insertion device of claim 1, wherein the button defines a cavity configured to at least partially enclose the needle hub in the depressed position.

3. The catheter insertion device of claim 2, wherein the body of the button comprises a first portion and a second portion, the first portion is positioned closer to the cavity than the second portion and has an opacity that is less than an opacity of the second portion.

4. The catheter insertion device of claim 2, wherein the needle hub comprises a base, a needle extending from the base, and an indicator that extends from the base opposite the needle, wherein the indicator is at least partially positioned within the cavity in the depressed position.

5. The catheter insertion device of claim 4, wherein:
the button is movable to an intermediate position between the raised position and the depressed position;
when moving from the raised position to the intermediate position, the needle moves with the button to extend out of the housing; and
when moving from the intermediate position to the depressed position, the needle retracts toward the body of the button to position the indicator within the cavity in the body.

6. The catheter insertion device of claim 5, further comprising a biasing member engaged with the needle hub to bias the needle hub toward the body of the button.

7. The catheter insertion device of claim 5, further comprising a catheter hub including a collar, a tooth extending from the collar, and a pin extending from the collar, the needle hub is selectively coupled to the catheter hub through engagement with the tooth to permit movement of the catheter hub with the needle hub and the button,
the pin is engaged with a slot defined by the housing that pivots the catheter hub when the catheter hub moves with the button between the raised position and the intermediate position, and
in the intermediate position, the catheter hub is pivoted relative to the needle hub to decouple the needle hub from the catheter hub, permitting the needle hub to move toward the body of the button.

8. The catheter insertion device of claim 7, wherein:
the catheter hub further comprises a catheter extending from the collar, the catheter surrounds the needle in the raised position and the intermediate position, where both the needle and the catheter are extended from the housing in the intermediate position, and
in the depressed position, the catheter is extended from the housing, and the needle is retracted into the housing with the needle hub positioned in the cavity of the body.

9. A catheter insertion device comprising:
a needle hub; and
a button movable relative to the needle hub and comprising a body defining a cavity configured to at least partially enclose the needle hub in the depressed position, and the body has an opacity that restricts visibility of the needle hub through the body when the needle hub is spaced apart from the cavity, and permits visibility of the needle hub through the body when the needle hub is at least partially positioned in the cavity.

10. The catheter insertion device of claim 9, wherein the body of the button comprises a first portion and a second portion, the first portion is positioned adjacent the cavity and has an opacity that is less than an opacity of the second portion.

11. The catheter insertion device of claim 9, wherein the needle hub comprises a base, a needle extending from the base, and an indicator that extends from the base opposite the needle, the indicator is at least partially positionable within the cavity.

12. The catheter insertion device of claim 11, wherein the button is movable from a raised position to an intermediate position, and from the intermediate position to a depressed position with the intermediate position between the raised position and the depressed position, when moving from the raised position to the intermediate position, the needle moves with the button to extend out of the housing, and when moving from the intermediate position to the depressed position, the needle retracts toward the body of the button to position the indicator within the cavity in the body.

13. The catheter insertion device of claim 12, further comprising a biasing member engaged with the needle hub to bias the needle hub toward the body of the button.

14. The catheter insertion device of claim 12, further comprising a catheter hub including a collar, a tooth extending from the collar, and a pin extending from the collar, the needle hub is selectively coupled to the catheter hub through engagement with the tooth to permit movement of the catheter hub with the needle hub and the button,
the pin is engaged with a slot defined by a housing enclosing the needle hub that pivots the catheter hub when the catheter hub moves with the button between the raised position and the intermediate position, and
in the intermediate position, the catheter hub is pivoted relative to the needle hub to decouple the needle hub from the catheter hub, permitting the needle hub to move toward the body of the button.

15. A catheter insertion device comprising:
a button comprising a body having an first portion and a second portion, the first portion has an opacity that is less than an opacity of the second portion; and
a needle hub movable relative to the button, the needle hub comprises an indicator that is visible through the second portion and not visible through the first portion when the needle hub is a first distance from the body of the button, and the indicator is visible through both the second portion and the first portion when the needle hub is a second distance from the body of the button, the second distance being less than the first distance.

16. The catheter insertion device of claim 15, wherein the body of the button defines a cavity complementary to the shape of the needle hub to be configured to at least partially enclose the indicator of the needle hub.

17. The catheter insertion device of claim 16, wherein the needle hub further comprises a base, and a needle extending from the base opposite the indicator.

18. The catheter insertion device of claim 16, wherein the button is movable between a depressed position, a raised position, and an intermediate position between the raised position and the depressed position, when moving from the raised position to the intermediate position, the needle moves with the button to extend out of the housing, and when moving from the intermediate position to the depressed position, the needle retracts toward the body of the button to position the indicator within the cavity in the body.

19. The catheter insertion device of claim 18, further comprising a catheter hub including a collar, a tooth extending from the collar, and a pin extending from the collar, the needle hub is selectively coupled to the catheter hub through engagement with the tooth to permit movement of the catheter hub with the needle hub and the button,
the pin is engaged with a slot defined by a housing enclosing the needle hub that pivots the catheter hub when the catheter hub moves with the button between the raised position and the intermediate position, and
in the intermediate position, the catheter hub is pivoted relative to the needle hub to decouple the needle hub from the catheter hub, permitting the needle hub to move toward the body of the button.

20. The catheter insertion device of claim 19, wherein:
the catheter hub further comprises a catheter extending from the collar, the catheter surrounds the needle in the raised position and the intermediate position, where both the needle and the catheter are extended from the housing in the intermediate position, and
in the depressed position, the catheter is extended from the housing, and the needle is retracted into the housing with the needle hub adjacent the body of the button.

21. A catheter insertion device comprising:
a housing;
a button comprising a body and at least one arm extending from the body into the housing to movably couple the button to the housing, the button is movable between a raised position and a depressed position; and
a needle hub movable relative to the button, the needle hub comprises an indicator that is visible through the body of the button in the depressed position.

22. The catheter insertion device of claim 21, wherein the indicator of the needle hub is only visible through the body of the button when the button is in the depressed position.
